(19) United States
(12) Patent Application Publication
(10) Pub. No.: US 2009/0118673 A1
(43) Pub. Date: May 7, 2009

(54) NEEDLE INJECTION CATHETER

(76) Inventors: Jerett Creed, Hermosa Beach, CA (US); Emerson Parin, Houston, TX (US); Knut Sauertaig, Porgen (DE); Juan Granada, Orangeburg, NY (US)

Correspondence Address: BELASCO, JACOBS & TOWNSLEY LLP;HOWARD HUGHES CENTER, 6100 CENTER DRIVE, SUITE 630, LOS ANGELES, CA, 90045, US

(21) Appl. No.: 12/138,201

(22) Filed: Jun. 12, 2008

Related U.S. Application Data
(60) Provisional application No. 61/002,065, filed on Nov. 7, 2007.

(51) Int. Cl. A61M 5/178

(52) U.S. Cl. 604/164.01

(57) ABSTRACT

A needle injection catheter includes a delivery tube that slidably surrounds a hypotube that has at least three hollow needle portions connected to and extending outwardly at its distal end. The needle portions are formed of resilient material and curve outwardly from the delivery tube. The ends of the needle portions are shaped to penetrate tissue when the hypotube is urged out of the delivery tube. A first sensor is located adjacent the distal end of the delivery tube and a second sensor is located adjacent the end of the needle portion. The sensors are electrically connected to first and second notification devices located adjacent a first end of the delivery tube. Radiopaque markers are affixed to the delivery tube to locate the tip using an enhanced imaging system. The markers are sized, shaped, positioned and of different densities to locate the tip both axially and radially within body structures.

### RELATED APPLICATION

This application claims priority from U.S Provisional Application Ser. No. 61/002,065, filed Nov. 7, 2007 and incorporates by reference the disclosure thereof.

### FIELD OF INVENTION

This invention relates to the field of stem cell therapies, and more specifically to apparatus and methods for safely injecting cells and therapeutic materials into heart chamber or other organ walls.

### BACKGROUND OF THE INVENTION

There has been a significant increase in the amount of research and funding in the area of stem cell based therapies for repairing and treating various diseases. More specifically, the number of clinical trials using cell based approaches to treat cardiac related diseases has tripled in the last few years. While much focus has been placed on the particular cell types and mechanisms of action within various tissue types and locations within the heart, very little has been developed with respect to the delivery of cells. Most of the work coming out of Europe has concentrated on the intra coronary route of administration. This has proved to have mixed effects in the acute timeframe (less than 10 days) but poor results in the chronic setting.

Recently, efforts have been placed on the intramyocardial delivery of cells directly into the heart muscle. This is accomplished by accessing the inside chamber of the left ventricle via a retrograde crossing of the aortic valve. Once inside the chamber, the physician will attempt to directly inject a needle-based catheter into the tissue of the endocardial surface and deliver a particular therapy (cell based or otherwise). These needle based catheters are tracked in the vasculature via standard x-ray fluoroscopy which provides only 2D visualization. This visualization is not optimal inside a 3D space particularly given the desire to deliver cells specifically to certain areas of interest.

Manipulating the catheter from outside the body with only a 2 dimensional understanding of the catheter tip movement is not adequate in the long run of cell based delivery locally to the inside chambers of the heart or elsewhere in the body. In addition, it creates safety issues due to applying excessive force on the tip of the catheter and making gross movements of the catheter without knowing the area within the ventricle where the catheter tip is located. This safety issue has become the number one issue for cell injection procedures. What is required is a catheter that is unable to perforate the ventricle or organ wall regardless of the force translated to the tip with the possibility of real time imaging of that catheter utilizing standard x-ray equipment.

What is proposed is a needle based injection catheter that has enhanced safety features such that it is extremely difficult to perforate the myocardium and has diagnostic features adding to the safety profile. In addition, a catheter that with minimal modifications can also be imaged and tracked via a standard single c-arm fluoroscopy system is also described briefly. Various medical devices have been developed to address injecting cells, drugs and other therapeutic means into organs of the body.

U.S. Pat. No. 7,087,040, issued to McGuckin, Jr. et al., discloses a surgical apparatus for delivering fluid to treat a lesion comprising a housing, an elongated member extending from the housing, and a plurality of tines positioned in the housing. Each of the tines has a lumen and at least one opening communicating with the lumen for delivering fluid to the lesion. An actuator is operatively associated with the tines and actuable to a first position to move the plurality of tines from a retracted position substantially within the elongated member to a first deployed position extending from the elongated member and actuable to a second position to move the plurality of tines from the first position to a second deployed position.

U.S. Patent Application No. 2006/0004325, published for Hamatake et al. is directed to multi-lumen catheters with improved tip configurations, including a triple-lumen catheter which may be useful for apheresis. In one variation, the catheter has three lumens with distal openings angularly spaced apart and staggered axially with respect to one another. In another variation, the catheter has two lumens exiting distally and one centrally positioned lumen exiting proximally. A third variation is a catheter with a single distal opening and two proximal openings. The staggered lumen openings along the axial length of the catheter may decrease recirculation while maximizing flow rates.

U.S. Patent Application No. 2005/0228452, published for Mourias et al. illustrates an apparatus for treating tissue that includes a flexible catheter including a proximal end, a distal end for introduction into a chamber of a heart, a transparent balloon carried by the distal end, an optical imaging assembly carried by the distal end for imaging tissue structures beyond the distal end through the balloon, and a needle deployable from the tubular member for penetrating the tissue structure to treat tissue. The apparatus may include a source of stems cells or other therapeutic and/or diagnostic agent coupled to the needle, a guide catheter advanceable over the needle for accessing a region beyond the tissue structure penetrated by the needle, and/or an energy probe deployable from the catheter for delivering electrical energy to tissue in the region beyond the tissue structure. The apparatus may be used to deliver stem cells into infracted tissue or for ablating heart tissue, e.g., from a trans-septal approach.

U.S. Pat. No. 6,302,870, issued to Jacobsen et al. disclose an apparatus for injecting fluids into the walls of blood vessels, body cavities, and the like, includes a plurality of laterally flexible needles disposed in a catheter for exit either out the distal end of the catheter or the catheter or through corresponding side openings in the catheter. In the latter case, the terminal ends of the needles would be curved laterally, with each terminal end being positioned in a respective side opening so that when the needles were moved forwardly in the catheter, the terminal ends of the needles would move laterally out the respective openings to pierce a vessel or cavity wall adjacent to which the catheter was positioned. Hilts positioned near the terminal ends of the needles serve to control the depth of penetration of the needles.

U.S. Patent Application No. 2006/0278248, published for Viswanathan and U.S. Patent Application No. 2007/0179492, published for Pappone are directed to a method of applying an electrode on the end of a flexible medical device to the surface of a body structure, the method including navigating the distal end of the device to the surface by orienting the distal end and advancing the device until the tip of the device contacts the surface and the portion of the device proximal to the end prolapses. Alternatively the pressure can be monitored with a pressure sensor, and used as an input in a feed back control to maintain contact pressure within a pre-determined range.

It is an objective of the present invention to provide a method and apparatus for injecting cells, drugs or other therapeutic agents into heart or other organ walls while minimizing the danger of penetrating those walls with the injection device. It is a further objective to provide a feedback system for the apparatus that will allow a physician to determine the point at which the distal end of the injection apparatus comes in contact with the organ wall. It is a still further objective of the invention to provide more detailed feedback to inform the physician of the point at which the injection needles of the apparatus contact the organ walls. It is yet a further objective to provide an apparatus that can be easily guided to the desired location within the body by means of 2 dimensional X-ray or related scanning technology. In is another objective of the invention that the location of the catheter is able to be tracked both laterally and radially within the body. Finally, it is an objective of the present invention to provide such apparatus that is durable, inexpensive and compatible with standard sterilization procedures.

While some of the objectives of the present invention are disclosed in the prior art, none of the inventions found include all of the requirements identified.

### SUMMARY OF THE INVENTION

The present invention addresses all of the deficiencies of prior art needle injection catheter inventions and satisfies all of the objectives described above.

(1) A needle injection catheter providing the desired features may be constructed from the following components. A delivery tube is provided. The delivery tube has a first end, a second end and a handle portion adjacent the first end. A hypotube is provided. The hypotube is sized and shaped to fit slidably within the delivery tube, has a proximal end, a distal end and at least three hollow needle portions connected to and extending outwardly at the distal end. The needle portions are formed of resilient material and curve outwardly from a central axis of the delivery tube and have distal ends shaped to penetrate tissue when the hypotube is urged toward the second end of the delivery tube. A first sensor is provided. The first sensor is located adjacent the second end of the delivery tube and electrically connected to a first notification device located adjacent the first end. At least one second sensor is provided. The second sensor is located adjacent the end of the needle portion and electrically connected to a second notification device located adjacent the first end of the delivery tube.

(2) In a variant of the invention, the first and second sensors detect electrical voltages associated with contact with bodily fluids, tissues and organ walls.

(3) In another variant, a microcircuit is electrically connected to the first and second sensors and to a power supply.

(4) In still another variant, the microcircuit and power supply are contained within the handle portion.

(5) In yet another variant, the first and second notification devices are either light emitting devices or sound emitting devices.

(6) In a further variant, the second notification device differs from the first notification device to indicate a hazard condition should the first sensor contact tissue.

(7) In still a further variant, extension of the needle portions from the second end of the delivery tube is limited to at least one predetermined distance by an extension control located adjacent the handle portion.

(8) In yet a further variant, the extension control provides for a predetermined minimum extension of the needle portions beyond the second end of said delivery tube.

(9) In another variant of the invention, the hypotube is connected to either a fixed luer connection or a flexible luer connection adjacent the handle portion to facilitate introduction of an injectate.

(10) In yet another variant, either a fixed or deflectable tip is located adjacent the second end of the delivery tube.

(11) In a further variant of the invention, a fixed tip is located adjacent the second end of the delivery tube. The fixed tip has an angled portion to facilitate perpendicular contact with the surface being injected,

(12) In yet a further variant, the second sensor detects voltage from at least one of the needle portions.

(13) In another variant, the needle injection catheter includes at least one radiopaque marker located either upon or within the delivery tube.

(14) In yet another variant, the radiopaque marker is located adjacent the second end of the delivery tube.

(15) In still another variant, the radiopaque markers are differentiated so that the location of each marker relative to other markers and the second end of the delivery tube is determined.

(16) In a final variant, the radiopaque markers are located circumferentially either upon or within the delivery tube and are tapered in form to indicate the radial position of the delivery tube when viewed with a radiological scanning system.

An appreciation of the other aims and objectives of the present invention and an understanding of it may be achieved by referring to the accompanying drawings and the detailed description of a preferred embodiment.

### DESCRIPTION OF THE DRAWINGS

FIG. 1 is a side elevational view of the preferred embodiment of the invention including a schematic representation of a microcircuit and power supply connected to first and second sensors and notification devices;

FIG. 2 is an enlarged, detailed side elevational view of the second end of the delivery tube of the FIG. 1 embodiment, illustrating the first sensor;

FIG. 3 is an enlarged, detailed side elevational view of a distal end of one of the needle portions of the FIG. 1 embodiment;

FIG. 4 is an enlarged, detailed side elevational view of the handle portion of the FIG. 1 embodiment illustrating the enclosed microprocessor and power supply; and

FIG. 5 is an enlarged, detailed side elevational view of the handle portion illustrating a fixed luer connection;

FIG. 6 is an enlarged, detailed side elevational view of a fixed tip of the delivery tube illustrating an angled portion;

FIG. 7 is an enlarged, detailed side elevational view of the tip illustrating radiopaque markers;

FIG. 8 is a front side elevation view of a man, illustrating the relative locations of the heart, aorta and femoral arteries and point of introduction of the needle injection catheter;

FIG. 9 is a partial cross-sectional view of a heart, illustrating the path for introduction of the catheter into the left ventricle of the heart; and

FIG. 10 is an enlarged view of the left ventricle illustrating the insertion of the needle portions into the heart wall.

### DETAILED DESCRIPTION OF THE PREFERRED EMBODIMENT

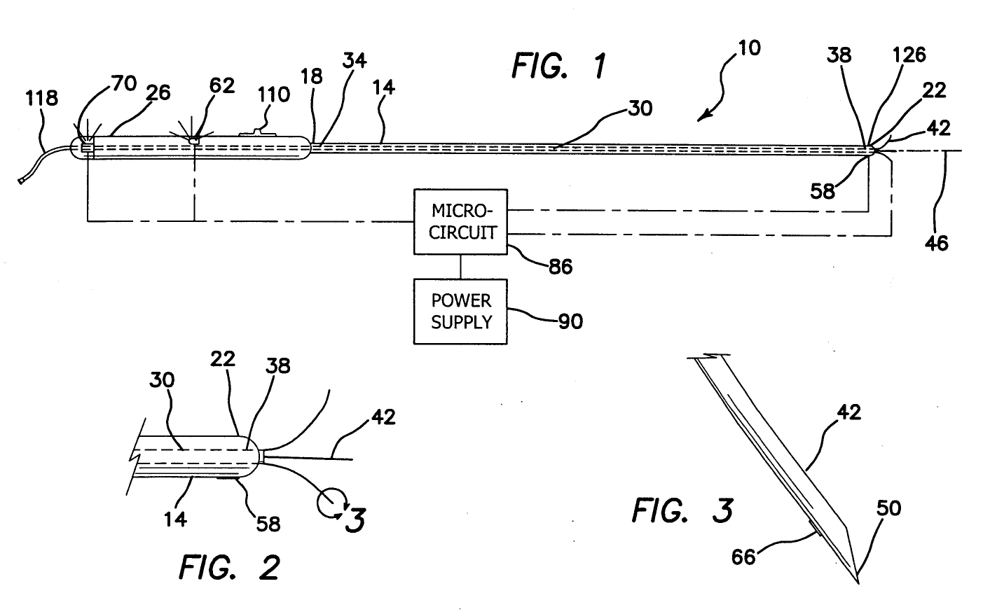

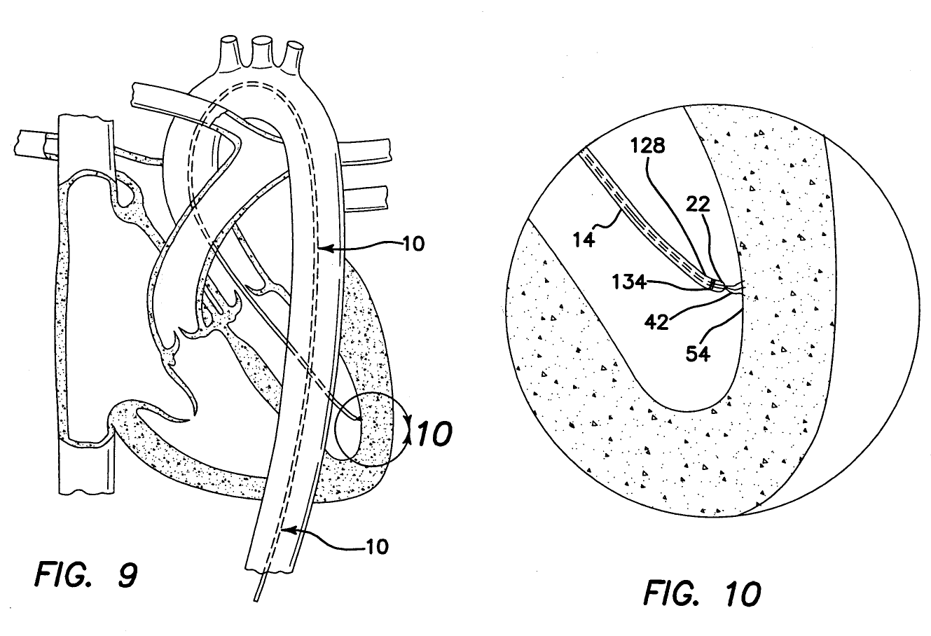

(1) FIGS. 1-10 illustrate a needle injection catheter 10 providing the desired features that may be constructed from the following components. As illustrated in FIGS. 1-3, a delivery tube 14 is provided. The delivery tube 14 has a first end 18, a second end 22 and a handle portion 26 adjacent the first end 18. A hypotube 30 is provided. The hypotube 30 is sized and shaped to fit slidably within the delivery tube 14, has a proximal end 34, a distal end 38 and at least three hollow needle portions 42 connected to and extending outwardly at the distal end 38. The needle portions 42, are formed of resilient material and curve outwardly from a central axis 46 of the delivery tube 14 and have distal ends 50 shaped to penetrate tissue 54 when the hypotube 30 is urged toward the second end 22 of the delivery tube 14. A first sensor 58 is provided. The first sensor 58 is located adjacent the second end 22 of the delivery tube 14 and electrically connected to a first notification device 62 located adjacent the first end 18. At least one second sensor 66 is provided. The second sensor 66 is located adjacent the end 50 of the needle portion 42 and electrically connected to a second notification device 70 located adjacent the first end 18 of the delivery tube 14.

(2) In a variant of the invention, the first 58 and second 66 sensors detect electrical voltages 74 associated with contact with bodily fluids 78, tissues 54 and organ walls 82.

(3) In another variant, a microcircuit 86 is electrically connected to the first 58 and second 66 sensors and to a power supply 90.

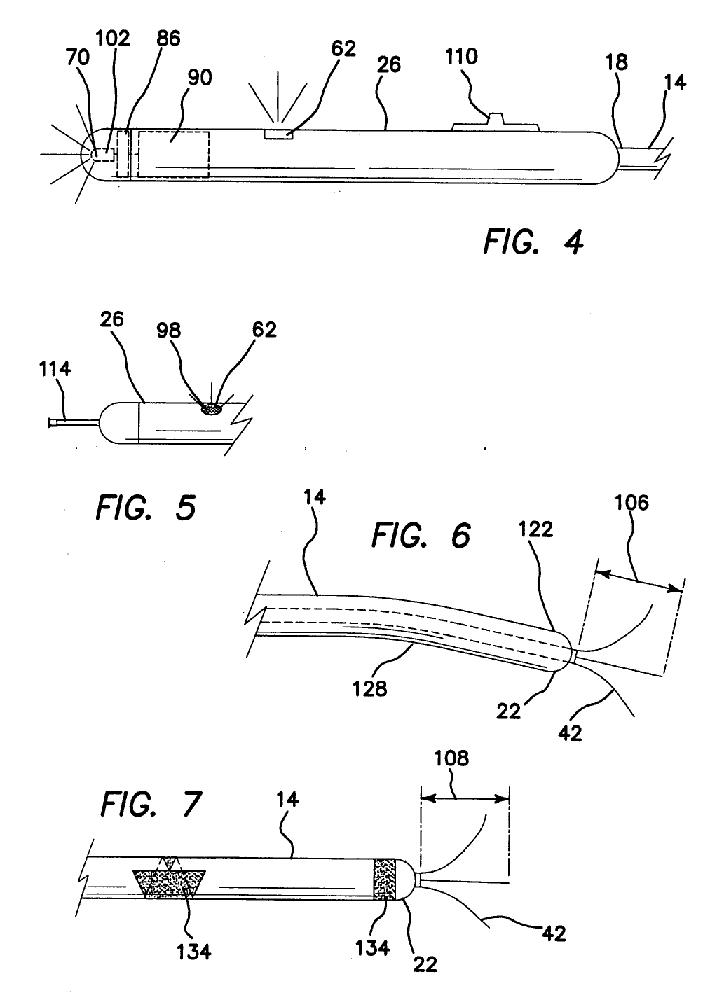

(4) In still another variant, as illustrated in FIG. 4, the microcircuit 86 and power supply 90 are contained within the handle portion 26.

(5) In still another variant, as illustrated in FIGS. 4 and 5, the first 58 and second 66 sensors detect changes in impedance 94 between bodily fluids 78, tissues 54 and organ walls 82.

(5) In yet another variant, the first 62 and second 70 notification devices are either light emitting devices 98 or sound emitting devices 102.

(6) In a further variant, the second notification device 70 differs from the first notification device 62 to indicate a hazard condition should the first sensor 58 contact tissue 54. See FIG. 10.

(7) In still a further variant, as illustrated in FIGS. 1, 4 and 6, extension of the needle portions 42 from the second end 22 of the delivery tube 14 is limited to at least one predetermined distance 106 by an extension control 110 located adjacent the handle portion 26.

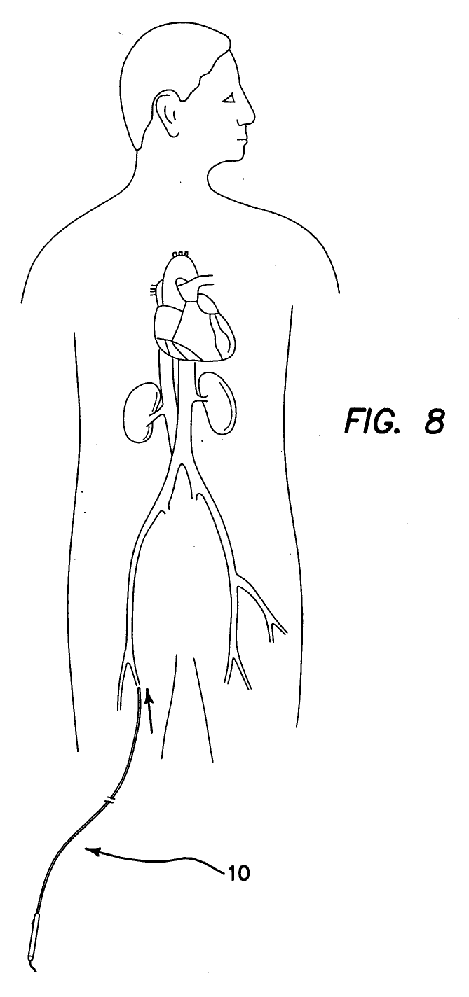

(8) In yet a further variant, as illustrated in FIG. 7, the extension control 110 provides for a predetermined minimum extension 108 of the needle portions 42 beyond the second end 22 of said delivery tube 14.

(9) In another variant of the invention, the hypotube 30 is connected to either a fixed luer connection 114 (FIG. 5) or a flexible luer connection 118 (FIG. 1) adjacent the handle portion 26 to facilitate introduction of an injectate (not shown).

(10) In yet a further variant, either a fixed 122 (FIG. 6) or deflectable tip 126 (FIG. 1) is located adjacent the second end 22 of the delivery tube 14.

(11) In a further variant of the invention, as illustrated in FIG. 6, a fixed tip 122 is located adjacent the second end 22 of the delivery tube 14. The fixed tip 122 has an angled portion 128 to facilitate perpendicular contact with a surface (not shown) being injected,

(12) In yet a further variant, the second sensor 66 detects voltage from at least one of the needle portions 42.

(13) In another variant, the needle injection catheter 10 includes at least one radiopaque marker 134 located either upon or within the delivery tube 14.

(14) In yet another variant, the radiopaque marker 134 is located adjacent the second end 22 of the delivery tube 14.

(15) In still another variant, the radiopaque markers 134 are differentiated so that the location of each marker 134 relative to other markers 134 and the second end 22 of the delivery tube 14 is determined.

(16) In a final variant, the radiopaque markers 134 are located circumferentially either upon or within the delivery tube 14 and are tapered in form to indicate the radial position of the delivery tube 14 when viewed with a radiological scanning system 138.

The needle injection catheter 10 has been described with reference to particular embodiments. Other modifications and enhancements can be made without departing from the spirit and scope of the claims that follow.

1. A needle injection catheter, comprising:
a delivery tube, said delivery tube having a first end, a second end and a handle portion adjacent said first end;
a hypotube, said hypotube being sized and shaped to fit slidably within said delivery tube, having a proximal end, a distal end and at least three hollow needle portions connected to and extending outwardly at said distal end;
said needle portions, being formed of resilient material, curving outwardly from a central axis of said delivery tube and having distal ends shaped to penetrate tissue when said hypotube is urged toward said second end of said delivery tube;
a first sensor, said first sensor being disposed adjacent said second end of said delivery tube and electrically connected to a first notification device disposed adjacent said first end; and
at least one second sensor, said second sensor being disposed adjacent said end of said needle portion and electrically connected to a second notification device disposed adjacent said first end of said delivery tube.

2. The needle injection catheter, as described in claim 1, wherein said first and second sensors detect electrical voltages associated with contact with bodily fluids, tissues and organ walls.

3. The needle injection catheter, as described in claim 2, further comprising a microcircuit electrically connected to said first and second sensors and to a power supply.

4. The needle injection catheter, as described in claim 3, wherein said microcircuit and power supply are contained within said handle portion.

5. The needle injection catheter, as described in claim 1, wherein said first and second notification devices are either of light emitting devices and sound emitting devices.

6. The needle injection catheter, as described in claim 5, wherein said second notification device differs from said first notification device to indicate a hazard condition should said first sensor contact tissue.

7. The needle injection catheter, as described in claim 1, wherein extension of said needle portions from said second end of said delivery tube is limited to at least one predetermined distance by an extension control disposed adjacent said handle portion.

8. The needle injection catheter, as described in claim 7, wherein said extension control provides for a predetermined minimum extension of said needle portions beyond said second end of said delivery tube.

9. The needle injection catheter, as described in claim 1, wherein said hypotube is connected to either of a fixed luer connection and a flexible luer connection adjacent said handle portion to facilitate introduction of an injectate.

10. The needle injection catheter, as described in claim 1, wherein either of a fixed and deflectable tip is disposed adjacent said second end of said delivery tube.

11. The needle injection catheter, as described in claim 1, wherein a fixed tip is disposed adjacent said second end of said delivery tube, said fixed tip having an angled portion to facilitate perpendicular contact with a surface being injected,

12. The needle injection catheter, as described in claim 3, wherein said second sensor detects voltage from at least one of said needle portions.

13. The needle injection catheter, as described in claim 1, further comprising at least one radiopaque marker disposed either of upon and within said delivery tube.

14. The needle injection catheter, as described in claim 14, wherein said radiopaque marker is disposed adjacent said second end of said delivery tube.

15. The needle injection catheter, as described in claim 14, wherein said radiopaque markers are differentiated so that the location of each marker relative to other markers and said second end of said delivery tube is determined.

16. The needle injection catheter, as described in claim 14, wherein said radiopaque markers are disposed circumferentially either of upon and within said delivery tube and are tapered in form to indicate the radial position of said delivery tube when viewed with a radiological scanning system.
